(19) United States
(12) Patent Application Publication
(10) Pub. No.: US 2025/0177781 A1
(43) Pub. Date: Jun. 5, 2025

(54) SYNCHRONIZATION CONTROL APPARATUS, SYNCHRONIZATION CONTROL METHOD, AND HEAVY PARTICLE BEAM IRRADIATION SYSTEM

(71) Applicants: KABUSHIKI KAISHA TOSHIBA, Tokyo (JP); TOSHIBA ENERGY SYSTEMS & SOLUTIONS CORPORATION, Kawasaki-shi (JP)

(72) Inventors: Ken MIYAHARA, Yokohama (JP); Ryuji SUGITO, Ota (JP); Yosuke SHIMIZU, Yokohama (JP); Chiayu LIN, Ota (JP); Tsubasa KUSAKA, Yokohama (JP); Kentaro MIYAZAKI, Yokohama (JP); Motojiro SHIBATA, Yokohama (JP); Yasushi OOISHI, Yokohama (JP); Tomio IWATA, Yokohama (JP)

(73) Assignees: KABUSHIKI KAISHA TOSHIBA, Tokyo (JP); TOSHIBA ENERGY SYSTEMS & SOLUTIONS CORPORATION, Kawasaki-shi (JP)

(21) Appl. No.: 18/829,527

(22) Filed: Sep. 10, 2024

(30) Foreign Application Priority Data
Dec. 5, 2023 (JP) 2023-205675

(51) Int. Cl. A61N 5/10

(52) U.S. Cl. CPC A61N 5/1048; A61N 5/1082; A61N 2005/1087

(57) ABSTRACT

According to one embodiment, a synchronization control apparatus comprising: at least two subordinate controllers configured to control at least two drive sources while feeding back respective displacement amounts measured by at least two displacement sensors in such a manner that at least two rotating frames reaches a position of a target rotation angle indicating an inputted target-position command value; and at least two superior controllers corresponding to respective subordinate controllers, each of the superior controllers being configured to correct the target-position command value by using a correction-value table and output a corrected target-position command value to a corresponding subordinate controller, the correction-value table being a table in which correction values for correcting the target-position command value are registered in advance.

### CROSS-REFERENCE TO RELATED APPLICATION

This application is based upon and claims the benefit of priority from Japanese Patent Application No. 2023-205675, filed on Dec. 5, 2023, the entire contents of which are incorporated herein by reference.

### TECHNICAL FIELD

Embodiments of the present invention relate to synchronization control technology.

### BACKGROUND

When two rotating frames of a certain gantry are moved by two drive shafts, though both drive shafts must be operated synchronously, there is a possibility that the stop position of each rotating frame deviates from its target command position due to factors such as machine differences between parts, machining errors of parts, and deformation of parts. Thus, it is necessary to prepare each target command position and a correction-value table corresponding to each target command position and to resolve the position error. However, even if feedforward control using the correction value is performed, the position error of each rotating frame may not be resolved due to aging degradation or time-dependent deterioration. Hence, it is necessary to periodically correct the correction-value table, which is time-consuming.

For example, in a known technique for a biaxial synchronization control apparatus, when only one axis is controlled and moved to a specified position, the position error between both axes is used as a correction value. This technique can suppress influence of external force, which interferes with moving portions due to misalignment of an encoder in full-axis positioning control as positioning control of each of both axes. However, in this technique, the correction value is set under the state where the other axis can be freely moved, and thus, this technique cannot be applied to a mechanism that cannot be moved by only one axis. From the viewpoint of driving only one side, the drive for updating the correction value must be performed separately from the actual operation. Further, the correction value is defined as the relative position error between both axes, and thus, desired positioning cannot be achieved if the absolute position of the main axis deviates from the command position due to equipment error.

In another known technique, each moving portion is moved to a predetermined position by biaxial synchronized operation, and then each moving portion is moved until achieving a predetermined attitude (i.e., posture) by independently moving the respective axes with the use of a sensor configured to detect the attitude of each moving portion. At this time, a command value newly given for correction is treated as the correction value, and positioning is performed during actual driving by adding this correction value to the position command value. This technique has only two measurement points for determining the correction value and is less accurate.

In still another technique for a multi-axis synchronization control apparatus, shafts are divided into a main shaft and a driven shaft, the relative position error between the main shaft and the driven shaft is set as the correction value, and positioning is performed by adding this correction value to the position command value. In this technique, the correction value is defined as the error in the relative position between the main shaft and the driven shaft, and thus, desired positioning cannot be achieved if the absolute position of the main shaft deviates from the command position due to equipment error.

In a heavy particle beam irradiation system having been widely used in recent years, a heavy irradiation port moves along arcuate rails. For example, one irradiation port is attached to two rotating frames that move along two parallel arcuate rails. The rotating frames are driven by a drive unit connected to both side surfaces.

Another possible mechanism is to use pin gears for driving the rotating frames. For example, it is possible to dispose a drive source only on the side surface of one of the rotating frames and cause a shaft to simultaneously rotate two pin gears that mesh with both side surfaces of the two rotating frames. However, this shaft interferes with the irradiation port that moves along with the rotating frames.

For this reason, in the drive mechanism of using two pin gears, it is conceivable to: dispose two drive sources on respective two rotating frames at their side surfaces; and simultaneously rotate both rotating frames by synchronously controlling the two pin gears meshing with each other on both side surfaces of the rotating frames. However, during drive of the rotating frames, there is a possibility that slight synchronization deviation between both drive sources causes positional deviation of the two rotating frames, and thus, a highly accurate positioning control technique is required.

The present invention aims to provide a technique that can synchronously drive two rotating frames for supporting a heavy load and perform positioning of both rotating frames with high precision at the time of driving both rotating frames.

### BRIEF DESCRIPTION OF THE DRAWINGS

In the accompanying drawings:

FIG. 1 is a schematic diagram illustrating an overall configuration of a heavy particle beam irradiation system;

FIG. 2 is a side view of a slit-type irradiator;

FIG. 3 is a front view of the slit-type irradiator;

FIG. 4 is a side view of a synchronization control apparatus according to the first embodiment;

FIG. 5 is a side view of the synchronization control apparatus in a state where each rotating frame is rotated upward;

FIG. 6 is a side view of the synchronization control apparatus in a state where each rotating frame is rotated downward;

FIG. 7 is a front view of the synchronization control apparatus taken along the line VII-VII of FIG. 4;

FIG. 8 is a block diagram illustrating the synchronization control apparatus according to the first embodiment;

FIG. 9 is a schematic diagram illustrating a correction-value table;

FIG. 10 is a block diagram illustrating the synchronization control apparatus according to the second embodiment;

FIG. 11 a conceptual diagram illustrating a state in which the rotating frames are misaligned;

FIG. 12 is a conceptual diagram illustrating a state in which misalignment of the rotating frames has been corrected;

FIG. 13 is a graph illustrating relationship between target-position command values before and after correction;

FIG. 14 is a flowchart illustrating synchronization control processing;

FIG. 15 is a flowchart illustrating angle-deviation detection processing;

FIG. 16 is a block diagram illustrating the synchronization control apparatus according to the third embodiment;

FIG. 17 is a conceptual diagram illustrating the rotating frames at the start of origin return;

FIG. 18 is a conceptual diagram illustrating the rotating frames in the state where the first origin-return has been completed;

FIG. 19 is a conceptual diagram illustrating the rotating frames in the state where the second origin-return has been completed;

FIG. 20 is a conceptual diagram illustrating the rotating frames during teaching of a control origin; and

FIG. 21 is a flowchart illustrating origin return processing.

### DETAILED DESCRIPTION

In one embodiment of the present invention, a synchronization control apparatus comprising: at least two arcuate rails fixedly provided in an arc-shape and arranged in such a manner that arcs of respective arcuate rails are parallel to each other; at least two rotating frames corresponding to the respective arcuate rails, each of the rotating frames being configured to rotationally move along a corresponding arcuate rail and around a center axis of the corresponding arcuate rail and support a support subject; at least two drive sources configured to provide torque for rotationally moving respective rotating frames; at least two torque transmitters provided in respective drive sources and configured to transmit the torque to the respective rotating frames; at least two displacement sensors provided in respective torque transmitters and configured to measure displacement amounts of respective components to be displaced by transmission of the torque, the respective components constituting the torque transmitters; at least two subordinate controllers configured to control the respective drive sources while feeding back respective displacement amounts measured by the displacement sensors in such a manner that each of the rotating frames reaches a position of a target rotation angle indicating an inputted target-position command value; and at least two superior controllers corresponding to respective subordinate controllers, each of the superior controllers being configured to correct the target-position command value by using a correction-value table and output a corrected target-position command value to a corresponding subordinate controller, the correction-value table being a table in which correction values for correcting the target-position command value are registered in advance.

### First Embodiment

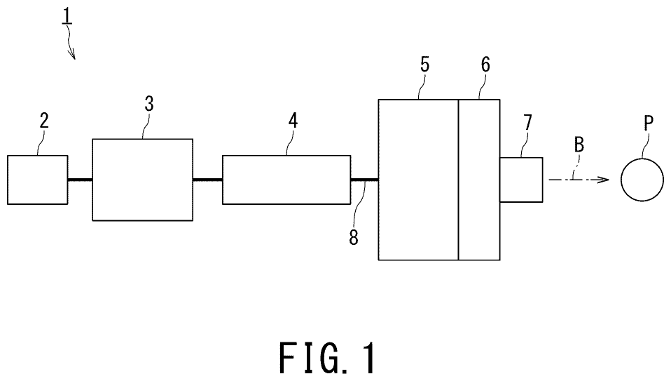

Hereinbelow, respective embodiments of a synchronization control apparatus, a synchronization control method, and a heavy particle beam irradiation system will be described in detail by referring to the accompanying drawings. The first embodiment will be described by using FIG. 1 to FIG. 9.

The reference sign 1 in FIG. 1 denotes a heavy particle beam irradiation system according to the present embodiment. The heavy particle beam irradiation system 1 is a so-called heavy-particle-beam cancer-treatment apparatus configured to perform treatment by irradiating a lesion tissue (i.e., cancerous site) of a patient P as an irradiation target with a heavy particle beam B, which is a beam using carbon ions as therapeutic radioactive rays, for example.

A radiation therapy with the use of the heavy particle beam irradiation system 1 like this is also referred to as a particle beam cancer treatment. This treatment is said to be able to damage the cancerous lesion (i.e., focus of disease) and minimize the damage to normal cells by pinpointing the cancerous lesion with carbon ions. Note that the heavy particle beam B is defined as radioactive rays heavier than a helium atom.

Although the heavy particle beam B using carbon ions is illustrated in the present embodiment, other aspects may be adopted. For example, helium, oxygen, or neon may be used as the heavy particle beam B.

As compared with the conventional cancer treatment using X-rays, gamma rays, or proton beams, the cancer treatment using the heavy particle beam B has characteristics that: (i) the ability to kill the cancerous lesion is higher; and (ii) the radiation dose is weak on the surface of the body of the patient P so as to peak at the cancerous lesion. Thus, the number of irradiations and side effects can be reduced, and the treatment period can be shortened.

The heavy particle beam B loses its kinetic energy at the time of passing through the body of the patient P so as to decrease its velocity and receive a resistance that is approximately inversely proportional to the square of the velocity and stops rapidly when it decreases to a certain velocity. The stopping point of the heavy particle beam B is referred to as the Bragg peak at which high energy is emitted. This Bragg peak is matched with the position of the lesion tissue of the patient, and thus, can kill only the lesion tissue while suppressing the damage to normal tissues.

The heavy particle beam irradiation system 1 includes an ion generator 2, an accelerator 3, a transportation apparatus 4, a slit-type irradiator 5, a synchronization control apparatus 6, and an irradiation port 7.

The ion generator 2 has an ion source of carbon ions, which are charged particles, and the heavy particle beam B is generated by these carbon ions. The accelerator 3 accelerates the heavy particle beam B generated by the ion generator 2. The accelerator 3 includes a linear accelerator and a circular accelerator. The heavy particle beam B is accelerated to about 70% of the speed of light while rotating around the circular accelerator about one million times. The heavy particle beam B accelerated by the circular accelerator is transported to the slit-type irradiator 5 by the transportation apparatus 4.

The ion generator 2, the accelerator 3, and the transportation apparatus 4 are provided with an integrally extended vacuum duct 8 (i.e., beam pipe), inside of which is vacuumized. The heavy particle beam B travels through the inside of this vacuum duct 8. This vacuum duct 8 forms a transport path that guides the heavy particle beam B from the ion generator 2 to the slit-type irradiator 5. In other words, the vacuum duct 8 is a sealed continuous space with sufficient degree of vacuum for allowing the heavy particle beam B to pass through.

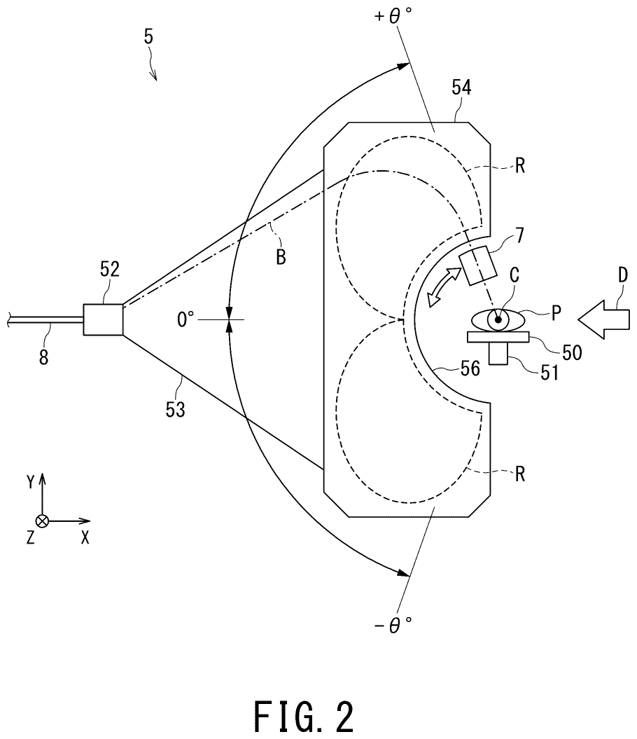

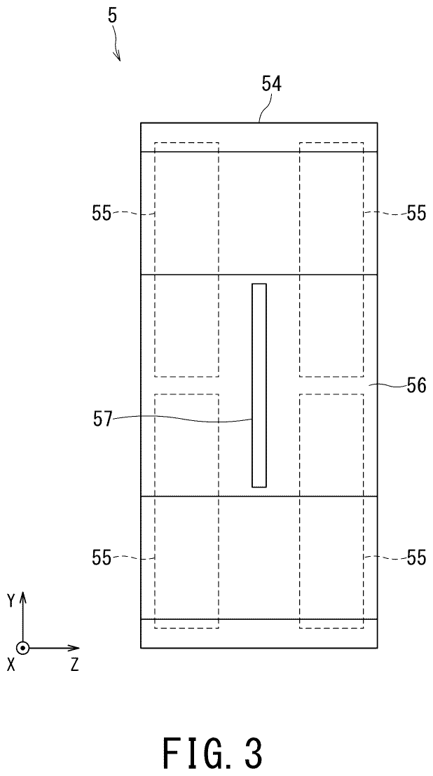

Next, the slit-type irradiator 5 will be described by referring to FIG. 2 and FIG. 3. Note that the right side of the sheet of FIG. 2 is assumed to be the front side (i.e., anterior side) of the slit-type irradiator 5. The direction along which the vacuum duct 8 of the transportation apparatus 4 extends and the heavy particle beam B circulates and comes flying is defined as the X-axis direction. The vertical direction perpendicular to this X-axis on the sheet of FIG. 2 is defined as the Y-axis direction, and the direction perpendicular to both the X-axis and the Y-axis is defined as the Z-axis direction.

First, a bending electromagnet 52 is provided at the end of the vacuum duct 8 of the transportation apparatus 4. An expansion duct 53 is provided so as to expand from this bending electromagnet 52 in a triangular shape or fan shape in lateral view. The expansion duct 53 expands in the Y-axis direction from the end of the vacuum duct 8 of the transportation apparatus 4. A main body 54 is connected to the tip of the expansion duct 53. The main body 54 forms a vertically long rectangular shape in lateral view. The inside of both the main body 54 and the expansion duct 53 is a sealed space in which the vacuum degree is kept continuous from the vacuum duct 8 of the transportation apparatus 4.

Inside of the main body 54 is provided with many bending electromagnets 55 (FIG. 3) that deflect the heavy particle beam B made incident from a wide angle range and focus the heavy particle beam B on an isocenter C. These bending electromagnets 55 generate at least one effective magnetic field region R (FIG. 2). The isocenter C is set as the position to be most convergently irradiated with the heavy particle beam B, and the lesion site of the patient P is placed at this isocenter C.

For example, inside the main body 54, a pair of bending electromagnets 55 arranged in the Z-axis direction are provided. Further, other two pairs of bending electromagnets 55 are arranged side by side in the Y-axis direction. One pair of the bending electromagnets 55 generate one effective magnetic field region R. In the case of FIG. 2, two pairs of bending electromagnets 55 arranged one above the other can generate two effective magnetic field regions R arranged one above the other.

Each effective magnetic field region R is formed to have a crescent shape in lateral view. The trajectory of the heavy particle beam B can be controlled by controlling the strength of each effective magnetic field region R. The heavy particle beam B can be radiated at an arbitrary angle, centered on the isocenter C. For example, when a tilt of a reference trajectory in the case of not deflecting the trajectory of the heavy particle beam B is set to 0°, the irradiation angle of the heavy particle beam B can be changed within a range from +θ° to −θ° centered on the isocenter C.

The reference trajectory is the trajectory in which the heavy particle beam B travels straight from the vacuum duct 8 to the isocenter C.

In the case of FIG. 2, the effective magnetic field regions R on the upper and lower sides are the same in shape and strength as each other. In other words, the two effective magnetic field regions R are formed in a vertically symmetrical manner, but other aspects may be adopted. For example, the effective magnetic field regions R may be vertically asymmetrical. In other words, the two effective magnetic field regions R on the upper and lower sides may be different in shape and strength from each other. Furthermore, it may be configured such that a total of only one effective magnetic field region R is formed on the upper or lower side. The center of the angle range of the heavy particle beam B to be changed in the circumferential direction around the isocenter C may deviate from the reference trajectory of the heavy particle beam B.

The patient P is placed on a movable table 50. This movable table 50 is supported by a movable arm 51, and moves with the patient P placed thereon so as to position the lesion site of the patient P at the isocenter C. The patient P can be moved to the irradiation position of the heavy particle beam B and positioning can be achieved by moving the movable table 50. Thus, the heavy particle beam B can be radiated onto to the lesion tissue of the patient P with optimal precision.

The front side of the main body 54 is formed as a semicircularly concave recess 56 in lateral view. The isocenter C is set at the center of the semicircle of the recess 56, and the patient P is placed at this isocenter C. The movable table 50 can position the patient P at the isocenter C by inserting the patient P into the recess 56 on the front side of the main body 54. For example, the movable table 50 with the patient P placed thereon can be inserted from the front of the slit-type irradiator 5 (in the direction of the white arrow D in FIG. 2). In this manner, the patient P can be placed at the isocenter C by approaching it from an appropriate direction.

A slit 57 is formed on the front side of the main body 54 so as to extend in the circumferential direction around the isocenter C where the patient P is placed. For example, a vertically long slit 57 (FIG. 3) is formed. The slit-type irradiator 5 emits the heavy particle beam B from the slit 57 toward the isocenter C at an arbitrary angle. The slit 57 is closed with an extremely heat-resistant and extremely cold-resistant polyimide film, and a vacuum is maintained inside the main body 54 in the state where the heavy particle beam B can pass through.

The irradiation port 7 is provided near the slit-type irradiator 5, and the irradiation port 7 can change the irradiation direction of the heavy particle beam B with respect to the isocenter C. The irradiation port 7 includes a ridge filter, a position monitor, a dose monitor, and a scanning magnet. In this manner, the irradiation port 7 is a heavy object with many instruments installed.

The irradiation port 7 moves circumferentially around the isocenter C, while being kept at an equal distance from the isocenter C where the patient P is placed.

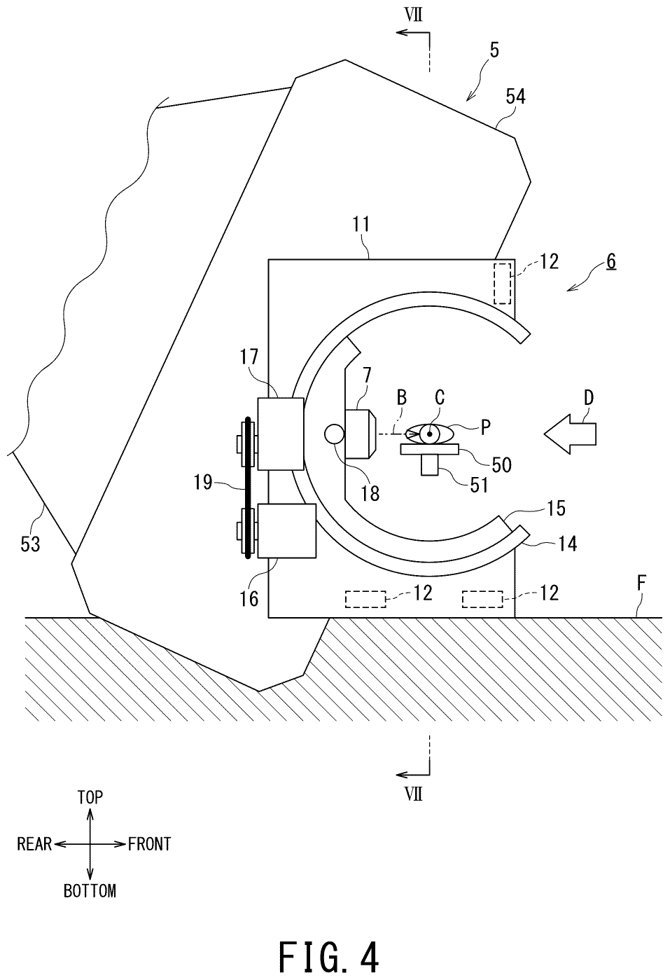

For example, when the tilt or inclination of the reference trajectory of the heavy particle beam B is set to 0°, the irradiation port 7 can be moved within the range from +θ° to −θ°. For example, the irradiation port 7 can be rotated in one of the circumferential directions and the opposite of the circumferential directions in increments of a predetermined angle. The irradiation port 7 moves along arcuate rails 14 (FIG. 4), which are C-shaped in lateral view and are provided on the synchronization control apparatus 6 (FIG. 4).

The irradiation port 7 moves along the contour (i.e., boundary shape) of the emission side of the effective magnetic field region R in lateral view. The heavy particle beam B traveling from the emission side of the effective magnetic field region R toward the isocenter C passes through the irradiation port 7, and the traveling direction of the heavy particle beam B is finely adjusted by the irradiation port 7.

In order to facilitate understanding in FIG. 2 and FIG. 3, the X-axis direction of the slit-type irradiator 5 is illustrated as a straight line that matches the horizontal direction. However, when the slit-type irradiator 5 is actually installed, the entire slit-type irradiator 5 is tilted as shown in FIG. 4. For example, the slit-type irradiator 5 is installed on a floor surface F in the state where the longitudinal direction (i.e., Y-axis direction) of its main body 54 is tilted.

In the present embodiment, the slit-type irradiator 5 is tilted in such a manner that the upper portion of the main body 54 faces toward the patient P. Although the irradiation range of the heavy particle beam B is within the range of any angle centered on the isocenter C, the tilt of the slit-type irradiator 5 enables irradiation of the heavy particle beam B from directly above the patient P.

In other words, the slit-type irradiator 5 is installed in a tilted state in such a manner that the reference trajectory in the case of not deflecting the trajectory of the heavy particle beam B by the slit-type irradiator 5 is tilted from the horizontal direction (i.e., horizontal axis). In this configuration, the range of angles at which the heavy particle beam B is radiated onto the patient P being the irradiation target of the heavy particle beam B becomes practical.

Although the upper portion of the main body 54 of the slit-type irradiator 5 is tilted toward the patient P in the present embodiment, the upper portion of the main body 54 may be tilted so as to be separated away from the patient P. Additionally, or alternatively, the slit-type irradiator 5 may be used without being tilted.

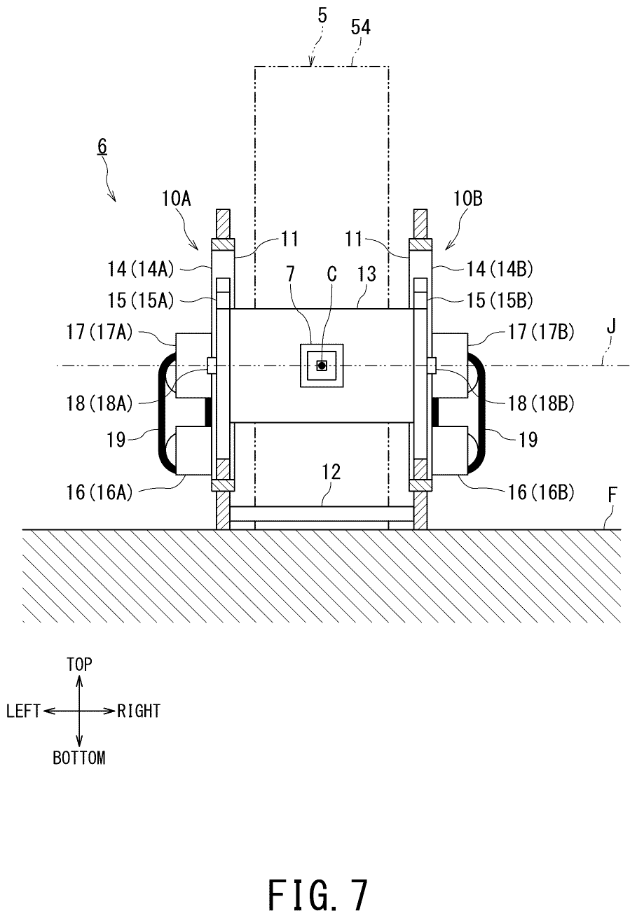

Next, the synchronization control apparatus 6 according to the first embodiment will be described by referring to FIG. 4 to FIG. 9. The synchronization control method is performed by using this synchronization control apparatus 6. In the following description, the right side of the sheet of FIG. 4 to FIG. 6 is assumed to be the front side (i.e., anterior side) of the synchronization control apparatus 6. In addition, the left side of the sheet of FIG. 7 is assumed to be the left side of the synchronization control apparatus 6, and the right side of the sheet of FIG. 7 is assumed to be the right side of the synchronization control apparatus 6.

As shown in FIG. 4, the synchronization control apparatus 6 is an apparatus for moving the irradiation port 7 in an arc along the inner circumferential surface of the recess 56 of the slit-type irradiator 5. The recess 56 is located on the front side of the main body 54 as shown in FIG. 2. The irradiation port 7 moves within the range where the slit 57 (FIG. 3) of the slit-type irradiator 5 is provided, and finely adjusts the heavy particle beam B to be emitted from the slit 57.

As shown in FIG. 7, the synchronization control apparatus 6 is a bilaterally symmetrical apparatus. The members and components disposed on the left side of the synchronization control apparatus 6 are collectively referred to as a first unit 10A, and the members and components disposed on the right side of the synchronization control apparatus 6 are collectively referred to as a second unit 10B. The first unit 10A and the second unit 10B have the same configuration. When the irradiation port 7 is moved, the first unit 10A and the second unit 10B are controlled so as to perform the same motion and operation and be synchronized with each other.

The synchronization control apparatus 6 has two support structures 11. These support structures 11 are wall-shaped, the bottom of each support structure 11 is fixed to the floor surface F, and these support structures 11 are erected side by side at a predetermined interval. A plurality of beam members 12 are suspended or bridged between the left and right support structures 11.

The synchronization control apparatus 6 further includes one port base 13, two arcuate rails 14, two rotating frames 15, two drive sources 16, two torque transmitters 17, and two angle sensors 18.

In the following description, the members and components corresponding to the first unit 10A are sometimes referred to as the first arcuate rail 14A, the first rotating frame 15A, the first drive source 16A, the first torque transmitter 17A, and the first angle sensor 18A. Similarly, the members and components corresponding to the second unit 10B are sometimes referred to as the second arcuate rail 14B, the second rotating frame 15B, the second drive source 16B, the second torque transmitter 17B, and the second angle sensor 18B.

The port base 13 is a plate-shaped member configured to support the irradiation port 7, which is a support subject or a portion to be supported. The irradiation port 7 is fixed at the center of the port base 13.

The arcuate rails 14 are fixedly provided by the support structures 11, and are arranged in an arc shape so that the arcs of both are parallel to each other.

The rotating frames 15 are guided by the respective arcuate rails 14 and rotate within a predetermined range of rotation angles around the center axis J (FIG. 7). The two left and right rotating frames 15 are fixed to the respective left and right sides of the port base 13. In other words, one irradiation port 7 is supported by the two rotating frames 15.

Note that the center axis J of the arcuate rails 14 extends horizontally and includes the isocenter C. The irradiation port 7 rotates around the isocenter C together with rotation of the rotating frames 15. The first rotating frame 15A and the second rotating frame 15B rotate around the same (single) center axis J.

Each drive source 16 is fixedly provided by the corresponding support structure 11, and generates torque for rotating the corresponding rotating frame 15 along the corresponding arcuate rail 14. The drive sources 16 are motors, for example.

The torque transmitters 17 are fixedly provided by the respective support structures 11 near the respective drive sources 16, and transmit the torque generated by the respective drive sources 16 to the respective rotating frames 15. In each of the first and second units 10A and 10B, the drive source 16 is connected to the torque transmitter 17 by a timing belt 19 for torque transmission.

The angle sensors 18 are provided in the respective rotating frames 15. Each angle sensor 18 detects the tilt of the rotating frame 15, i.e., the rotation angle when the rotating frame 15 rotates. When the rotating frames 15 rotate (move) along the respective arcuate rails 14, the position of each rotating frame 15 on the arcuate rail 14 can be determined from the rotation angle detected by the corresponding angle sensor 18. In the following description, the rotation angle detected by each angle sensor 18 is sometimes referred to as the absolute position of the rotating frame 15.

As shown in FIG. 4, each arcuate rail 14 and each rotating frame 15 form a C-shape with a part of the circle cut out in lateral view. The phrase “in lateral view” means viewing the synchronization control apparatus 6 from a direction that intersects the front-and-rear direction, and means viewing the synchronization control apparatus 6 from a direction that matches the extending direction of the center axis J. The opening dimensions of the cutout parts of each arcuate rail 14 and each rotating frame 15 are set in such a manner that the patient P can enter from a direction intersecting (i.e., perpendicular to) the center axis J of the arcuate rails 14 at the time of placing the patient P near the irradiation port 7. In this configuration, the patient P can be placed at the isocenter C on the center axis J of the arcuate rails 14 by inserting the patient P from an appropriate direction (e.g., the direction of the white arrow D in FIG. 4).

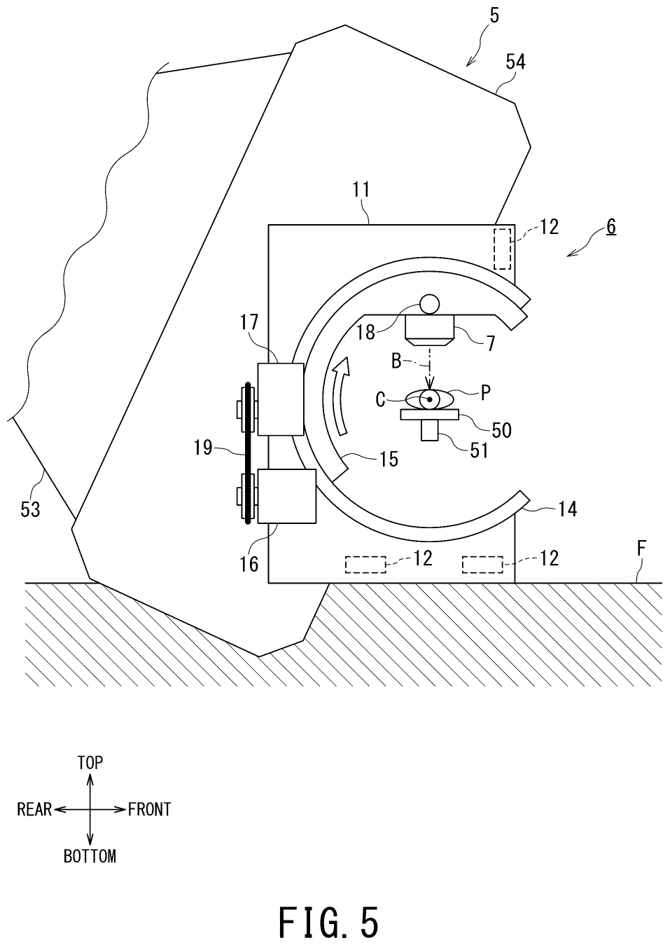

As shown in FIG. 5, when the irradiation port 7 is moved above the patient P in order to radiate the heavy particle beam B from above the patient P, the rotating frames 15 rotate (move) upward along the respective arcuate rails 14.

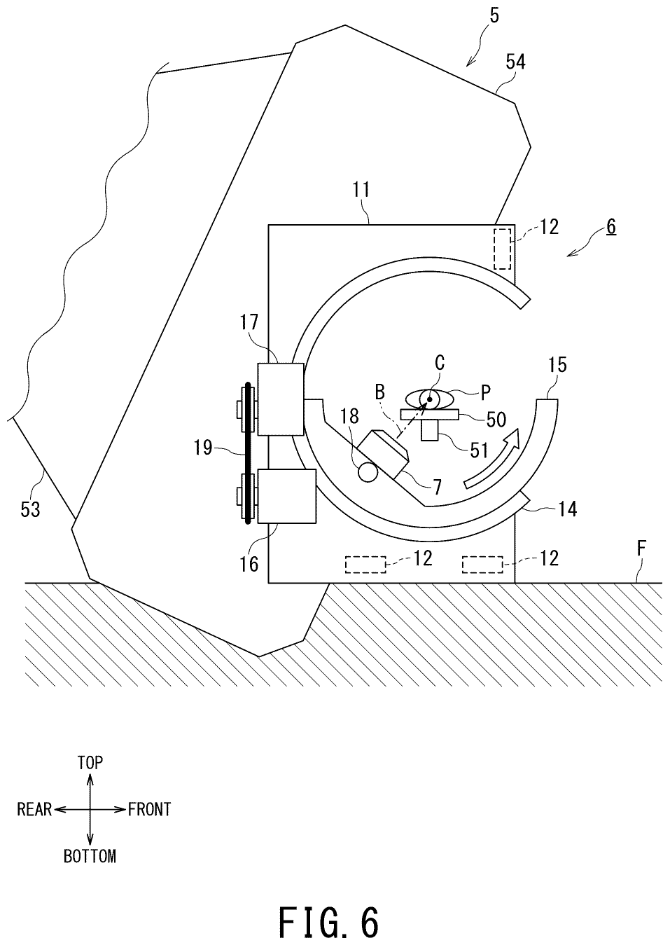

As shown in FIG. 6, when the irradiation port 7 is moved obliquely downward in order to radiate the heavy particle beam B from the obliquely downward direction of the patient P, the rotating frames 15 rotate (move) downward along the respective arcuate rails 14.

As shown in FIG. 4, the plurality of beam members 12 connecting the left and right support structures 11 are provided at the front portion and the bottom portion of the support structures 11. Between the left and right support structures 11, the main body 54 of the slit-type irradiator 5 is provided. The main body 54 is disposed at the rear of the support structures 11. Furthermore, the vertical dimensions of the main body 54 are larger than the vertical dimensions of the support structures 11.

Since the main body 54 of the slit-type irradiator 5 is disposed at the rear of the support structures 11, the beam members 12 connecting the left and right support structures 11 cannot be provided at the rear of the support structures 11. Thus, it is difficult to ensure rigidity of the rear of the support structures 11. In this case, for example, correction is required such that misalignment is not caused at the stop positions of the respective rotating frames 15.

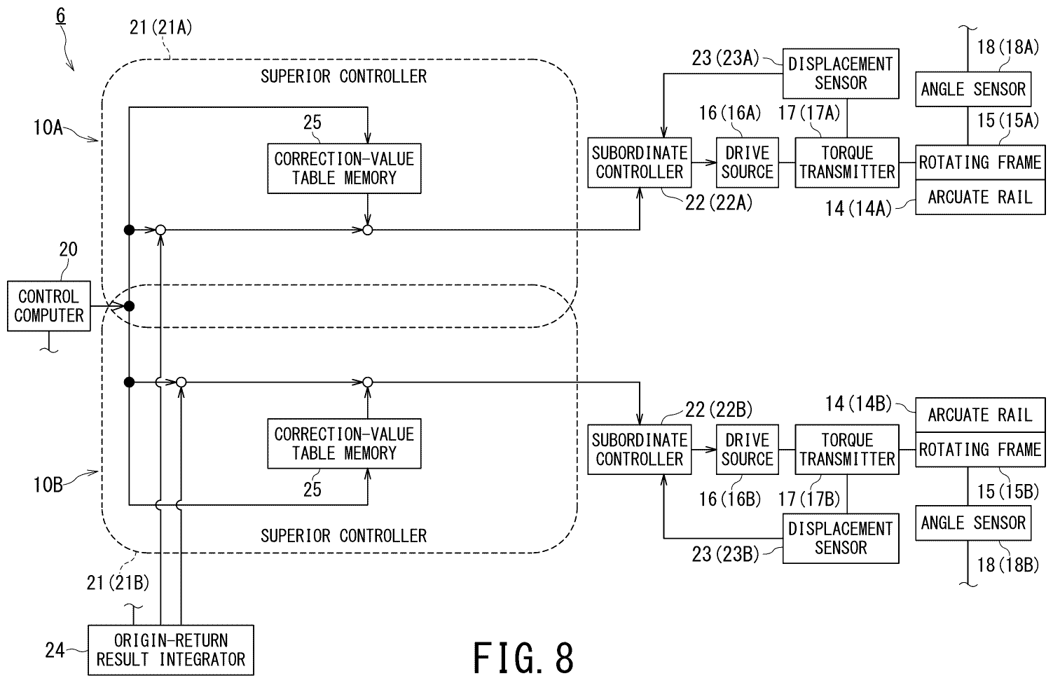

Next, the system configuration of the synchronization control apparatus 6 will be described by referring to the block diagram of FIG. 8. Each arrow in FIG. 8 is only one interpretation for illustrating the flow of data including a predetermined value or signal, and a flow of data excluding the arrows in this diagram may be included. Further, the anteroposterior relationship of the respective steps is not necessarily fixed, and the anteroposterior relationship of one or more steps may be replaced or changed. Moreover, two or more of the steps may be executed in parallel with each other. Further, the synchronization control apparatus 6 may include configurations excluding the components shown in FIG. 8 or one or more components of the synchronization control apparatus 6 shown in FIG. 8 may be omitted.

In addition to the above-described components, the synchronization control apparatus 6 further includes one control computer 20, two superior controllers 21, two subordinate controllers 22, two displacement sensors 23, and one origin-return result integrator 24.

In the following description, the components corresponding to the first unit 10A are sometimes referred to as the first superior controller 21A, the first subordinate controller 22A, and the first displacement sensor 23A. Similarly, the components corresponding to the second unit 10B are sometimes referred to as the second superior controller 21B, the second subordinate controller 22B, and the second displacement sensor 23B.

The control computer 20 is a device that controls the synchronization control apparatus 6 in response to a user's input operation. For example, the control computer 20 outputs a target-position command value to each superior controller 21 in response to the user's input operation. The target-position command value is a value indicative of a rotation angle (i.e., absolute position) which each rotating frame 15 is aimed to reach.

Each angle sensor 18 is connected to the control computer 20 and sends the measured rotation angle of the corresponding rotating frame 15 to the control computer 20. On the basis of the rotation angles detected by the respective angle sensors 18, the control computer 20 can determine whether the rotating frames 15 have reached the target-position command value or not.

Each superior controller 21 includes a correction-value table memory 25 configured to store a correction-value table in which correction values for correcting the target-position command value are registered in advance. Each superior controller 21 corrects the target-position command value on the basis of the correction-value table stored in the correction-value table memory 25, and outputs the corrected target-position command value to the corresponding subordinate controller 22.

Each superior controller 21 includes hardware resources such as a processor and a memory and is configured as a computer in which information processing based on software is achieved with the use of the hardware resources by causing its a Central Processing Unit (CPU) to execute various programs. Each superior controller 21 is composed of a Programmable Logic Controller (PLC), for example. Further, the synchronization control method according to the present embodiment is achieved by causing the superior controllers 21 configured as computers to execute various programs.

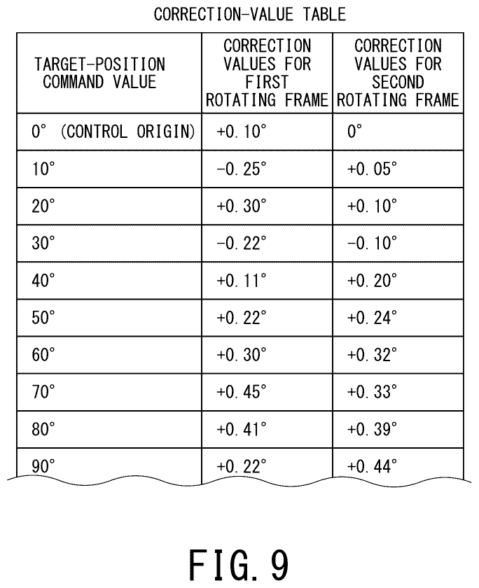

As shown in FIG. 9, in the correction-value table, respective correction values are registered in association with target-position command values, which are respective rotation angles when the corresponding rotating frame 15 rotates. The correction values corresponding to the first rotating frame 15A and the correction values corresponding to the second rotating frame 15B are registered in the correction-value table for every 10 degrees of rotation. In order to facilitate understanding in FIG. 9, one correction-value table stored by the first superior controller 21A and the other correction-value table stored by the second superior controller 21B are collectively illustrated as a single correction-value table.

Each correction value indicates a rotation angle for correction required to make the rotating frame 15 reach the target position when the rotating frame 15 does not reach the target position due to factors such as distortion of the arcuate rail 14, processing errors, and deformation caused by aging degradation.

For example, it is assumed that “10°” is inputted as the target-position command value into the first superior controller 21A and the second superior controller 21B from the control computer 20. In this case, on the basis of the correction-value table, the first superior controller 21A outputs a value obtained by adding “−0.25°” to “10” toward the first subordinate controller 22A, whereas the second superior controller 21B outputs a value obtained by adding “+0.05°” to “10” toward the second subordinate controller 22B.

In the present embodiment, “addition” includes an aspect of adding a negative value. For example, “addition” includes an aspect of subtracting an absolute value of a predetermined value.

In addition, when the correction value corresponding to the target-position command value is not registered in the correction-value table, an interpolated value calculated from values around the target-position command value may be used. For example, it is assumed that “15°” is inputted as the target-position command value into the first superior controller 21A and the second superior controller 21B from the control computer 20. Under this assumption, the correction value corresponding to the target-position command value of “15°” is not registered in the correction-value table. Thus, the first superior controller 21A calculates the correction value (i.e., interpolated value) as “+0.025°”, which is the intermediate value between “−0.25°” registered as the correction value for “10°” and “+0.30°” registered as the correction value for “20°”. Similarly, the second superior controller 21B calculates the correction value (i.e., interpolated value) as “+0.075°”, which is the intermediate value between “+0.05°” registered as the correction value for “10°” and “+0.10” registered as the correction value for “20°”.

As shown in FIG. 8, each subordinate controller 22 controls the corresponding drive source 16 on the basis of the inputted target-position command value. The subordinate controllers 22 are drivers for the respective drive sources 16, for example. The target-position command value corrected by each superior controller 21 is inputted into the corresponding subordinate controller 22.

Each displacement sensor 23 is provided in the corresponding torque transmitter 17 and measures displacement of at least one component, which constitutes this torque transmitter 17 and is displaced by transmission of torque. Each displacement sensor 23 is a resolver built into the torque transmitter 17, for example. Each displacement sensor 23 measures rotation amount of a predetermined component, such as a transmission shaft (not shown) of the corresponding torque transmitter 17.

Each subordinate controller 22 controls the corresponding drive source 16 so as to make the corresponding rotating frame 15 reach the position of the target rotation angle, which is the position of the inputted target-position command value, while feeding back the displacement amount measured by the corresponding displacement sensor 23.

For example, the measurement value (e.g., displacement amount) of the first displacement sensor 23A of the first torque transmitter 17A is inputted into the first subordinate controller 22A. The first subordinate controller 22A determines the movement amount of the first rotating frame 15A on the basis of the inputted measurement value, and drives the first drive source 16A until the first rotating frame 15A reaches the target position on the first arcuate rail 14A.

Similarly, the measurement value (e.g., displacement amount) of the second displacement sensor 23B of the second torque transmitter 17B is inputted into the second subordinate controller 22B. The second subordinate controller 22B determines the movement amount of the second rotating frame 15B on the basis of the inputted measurement value, and drives the second drive source 16B until the second rotating frame 15B reaches the target position on the second arcuate rail 14B.

The origin-return result integrator 24 performs processing of adding an origin-offset value to the target-position command value, when origin offset is performed by the user to set an arbitrarily settable control origin at a position different from the mechanical origin specific to the apparatus. In this manner, the origin-offset value can be reflected in the target-position command value, and accurate positioning of the rotating frames 15 can be performed. The origin-return result integrator 24 is connected to the control computer 20 and is controlled by the control computer 20.

The origin “0°” registered as the target-position command value in the correction-value table (FIG. 9) is the control origin. When the origin-offset value is not reflected in the correction value, each superior controller 21 adds the origin-offset value to the correction value. Each superior controllers 21 outputs the corrected target-position command value to the corresponding subordinate controller 22.

Each subordinate controller 22 controls the corresponding drive source 16 on the basis of the corrected target-position command value so as to cause the corresponding rotating frame 15 to reach the position of the target rotation angle. Since each drive source 16 is controlled on the basis of the corrected target-position command value, each rotating frame 15 can be stopped at the correct position.

In the first embodiment, when both rotating frames 15 for supporting the heavy irradiation port 7 are driven, both rotating frames 15 can be driven synchronously, and the irradiation port 7 can be positioned with high accuracy. Thus, the heavy particle beam B can be precisely radiated to the lesion site of the patient P from the irradiation port 7.

### Second Embodiment

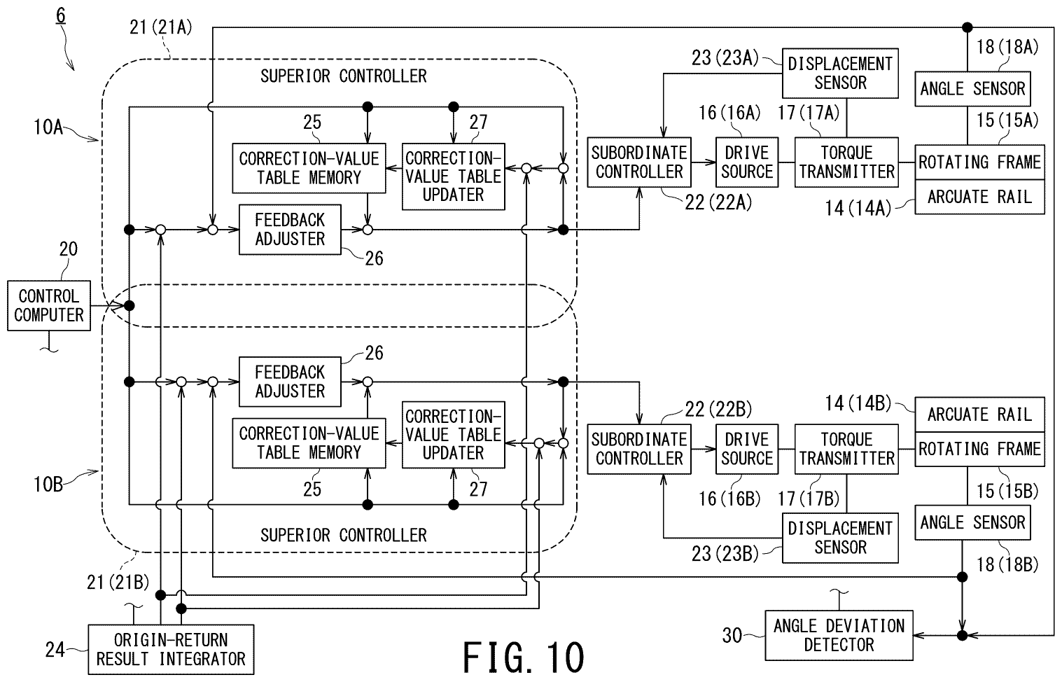

Next, the second embodiment will be described by referring to FIG. 10 to FIG. 15. The same components as those shown in the above-described embodiment are denoted by the same reference signs, and duplicate descriptions are omitted.

As shown in FIG. 10, each superior controller 21 according to the second embodiment includes a feedback adjuster 26 and a correction-value table updater 27 in addition to the components of the above-described first embodiment. Furthermore, the synchronization control apparatus 6 according to the second embodiment includes an angle deviation detector 30 in addition to the components of the above-described first embodiment.

Each feedback adjuster 26 determines whether there is a difference (i.e., first difference) between the rotation angle targeted by the target-position command value and the actual rotation angle (i.e., absolute position) detected by the corresponding angle sensor 18 or not. Each feedback adjuster 26 adjusts the target-position command value of the corresponding rotating frame 15 in such a manner that this difference falls within a predetermined range (i.e., first threshold). On the basis of the adjusted target-position command value when the difference (i.e., first difference) falls within this predetermined range, the correction-value table updater 27 updates the correction value registered in the correction-value table of the corresponding rotating frame 15. This control can adjust the difference (i.e., first difference) caused by factors such as aging degradation, and can automatically update the correction-value table.

While the rotating frames 15 are rotating, the angle deviation detector 30 calculates the difference (i.e., second difference) in rotation angle between the first rotating frame 15A and the second rotating frame 15B on the basis of the respective rotation angles (i.e., absolute positions) detected by the first angle sensor 18A and the second angle sensor 18B. When this difference exceeds a predetermined threshold (i.e., second threshold), the angle deviation detector 30 stops the rotation of the first rotating frame 15A and the second rotating frame 15B. This control allows the first rotating frame 15A and the second rotating frame 15B to be safely stopped if a malfunction occurs in rotation of at least one of the first rotating frame 15A and the second rotating frame 15B. The angle deviation detector 30 is connected to the control computer 20 and is controlled by the control computer 20. The threshold values (i.e., the first threshold and the second threshold) are set in advance by the user.

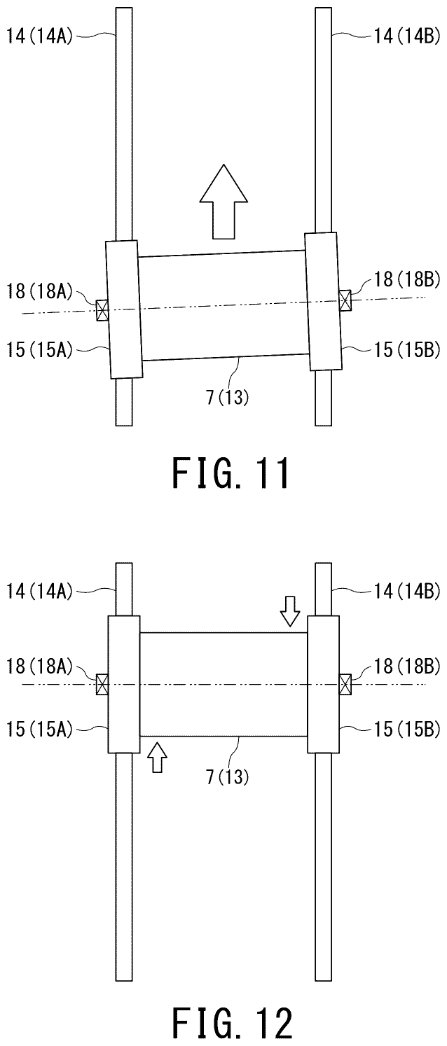

As shown in FIG. 11, the first angle sensor 18A is fixed to the first rotating frame 15A, and the second angle sensor 18B is fixed to the second rotating frame 15B. In some cases, the rotation angle (i.e., absolute position) detected by the first angle sensor 18A differs from the rotation angle (i.e., absolute position) detected by the second angle sensor 18B. Since the target-position command value is one specific rotation angle, if the rotation angles of the left and right rotating frames 15 are different from each other, the above-described difference (i.e., the first difference or the second difference) exists in either or both of the rotating frames 15.

First, each feedback adjuster 26 adjusts the target-position command value of the corresponding rotating frame 15 on the basis of the rotation angle measured by the corresponding angle sensor 18 such that the difference (i.e., the first difference) falls within the predetermined range. For example, as shown in FIG. 12, this adjustment is performed such that rotation of the first rotating frame 15A being insufficient in rotation amount is increased and rotation of the second rotating frame 15B having rotated too much is suppressed. Each feedback adjuster 26 adjusts or corrects the target-position command value such that the first difference is eliminated by this adjustment. The arbitrary range within which the difference (i.e., the first difference) must fall is set by the user in advance.

When the adjustment by each feedback adjuster 26 is successful, each correction-value table updater 27 updates the correction-value table. For example, each correction-value table updater 27 calculates the adjustment amount of the correction value adjusted by the corresponding feedback adjuster 26 on the basis of the uncorrected target-position command value inputted from the control computer 20 and the target-position command value adjusted by the corresponding feedback adjuster 26. Each correction-value table updater 27 adds the calculated adjustment amount to the corresponding correction value in the correction-value table stored in the correction-value table memory 25, and registers it as a new correction value in the correction-value table. This updated correction-value table is stored in each correction-value table memory 25.

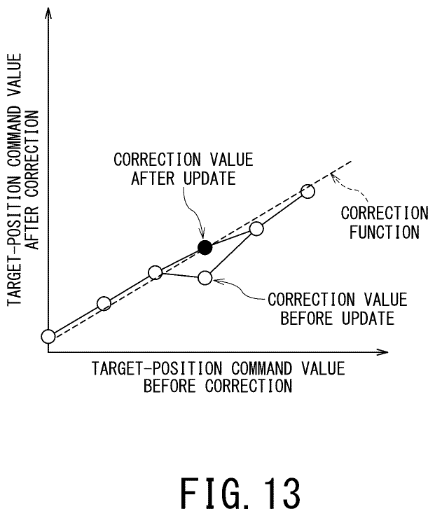

FIG. 13 is a graph of an approximate function that schematically shows relationship between the target-position command value before correction and the target-position command value after correction (i.e., the amount corrected by the correction value). As shown in this graph, when there are a plurality of correction values, the relationship between the target-position command values before and after correction can be expressed by a certain correction function. If there is a correction value that deviates significantly from this correction function, this correction value may not be correct. The correction value updated by each correction-value table updater 27 approaches this correction function. In addition, each correction-value table updater 27 may perform the processing of updating the correction-value table in such a manner that the correction value approaches the correction function.

If the adjustment by the feedback adjusters 26 is not successful, the angle deviation detector 30 stops rotation of both rotating frames 15. For example, when the difference (i.e., the second difference) in rotation angle between the first rotating frame 15A and the second rotating frame 15B exceeds a predetermined threshold, the angle deviation detector 30 stops rotation of both rotating frames 15.

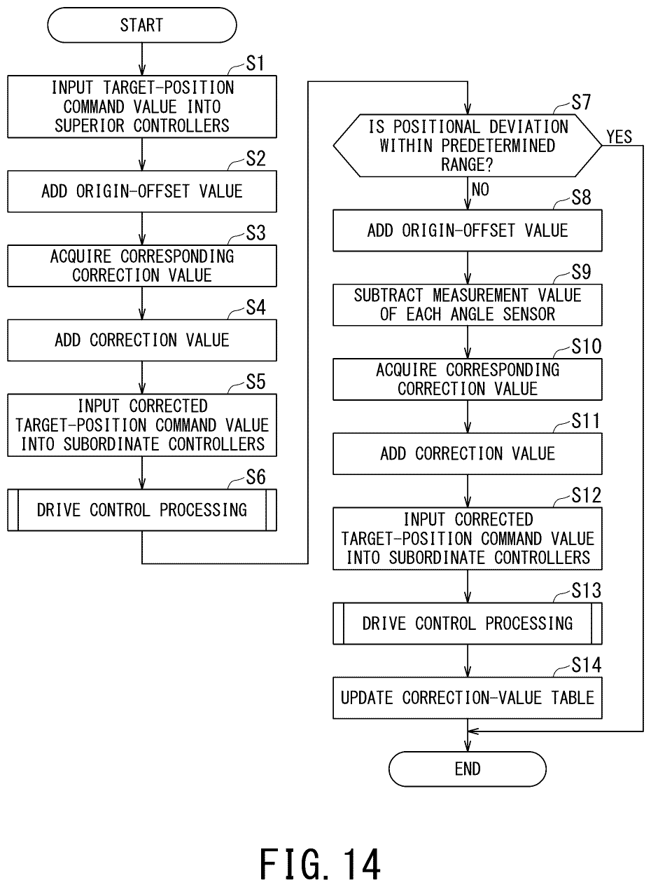

Next, synchronization control processing will be described on the basis of the flowchart of FIG. 14 by referring to the block diagram of FIG. 10. The following steps are at least part of the synchronization control processing, and other steps may be included in the synchronization control processing.

In the first step S1, the control computer 20 inputs the target-position command value into the superior controllers 21. The target-position command value at this time is the value before correction.

In the next step S2, the origin-return result integrator 24 adds the origin-offset value to the target-position command value inputted from the control computer 20.

In the next step S3, each superior controller 21 acquires the corresponding correction value on the basis of the correction-value table stored in the corresponding correction-value table memory 25.

In the next step S4, each superior controller 21 adds the acquired correction value to the target-position command value to which the origin-offset value has already been added.

In the next step S5, the corrected target-position command value is inputted from each superior controller 21 into the corresponding subordinate controller 22.

In the next step S6, the subordinate controllers 22 perform drive control processing. Each subordinate controller 22 controls the corresponding drive source 16 on the basis of the corrected target-position command value, and continues the drive control processing until the corresponding rotating frame 15 reaches the position indicated by the target-position command value. Each subordinate controller 22 completes the drive control processing when the corresponding rotating frame 15 reaches the position indicated by the target-position command value.

In the next step S7, each feedback adjuster 26 determines whether the first difference between the rotation angle (i.e., position) targeted by the target-position command value and the actual rotation angle (i.e., absolute position) detected by the corresponding angle sensor 18 is within the predetermined range (i.e., first threshold) or not. If the difference is within the predetermined range (YES in the step S7), the synchronization control processing is completed. If the difference is not within the predetermined range (NO in the step S7), the processing proceeds to the step S8.

In the next step S8, the origin-return result integrator 24 adds the origin-offset value to the target-position command value inputted from the control computer 20.

In the next step S9, into the corresponding feedback adjuster 26, each superior controller 21 inputs a value obtained by subtracting (or adding) the measurement value (i.e., rotation angle) of the angle sensor 18 of the corresponding rotating frame 15 from (or to) the target-position command value to which the origin-offset value has been added. When the value to be inputted into the feedback adjuster 26 contains the origin-offset value, in order to avoid double addition of the origin-offset value, the origin-offset value is subtracted from the value to be inputted.

In the next step S10, on the basis of the correction-value table stored in the corresponding correction-value table memory 25, each superior controller 21 acquires the correction value corresponding to the target-position command value to which the origin-offset value has been added.

In the next step S11, each superior controller 21 adds the acquired correction value to the target-position command value, from/to which the measurement value (i.e., rotation angle) of the corresponding angle sensor 18 has been subtracted/added.

In the next step S12, the corrected target-position command value is inputted from each superior controller 21 to the corresponding subordinate controller 22.

In the next step S13, the subordinate controllers 22 perform the drive control processing again. The subordinate controllers 22 complete the drive control processing when the rotating frames 15 reach the position indicated by the target-position command value.

In the next step S14, on the basis of the correction value newly generated from the step S8 to the step S12, each correction-value table updater 27 updates the correction value(s) registered in the correction-value table of the corresponding rotating frame 15, and then, the synchronization control processing is completed.

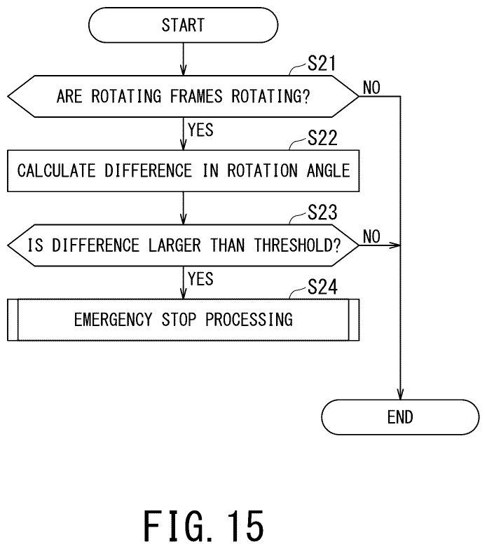

Next, angle-deviation detection processing will be described on the basis of the flowchart of FIG. 15 by referring to the block diagram of FIG. 10. The following steps are at least part of the angle-deviation detection processing, and other steps may be included in the angle-deviation detection processing.

In the first step S21, the angle deviation detector 30 determines whether the rotating frames 15 are rotating or not. If the rotating frames 15 are not rotating (NO in the step S21), the angle-deviation detection processing is completed. If the rotating frames 15 are rotating (YES in the step S21), the processing proceeds to the step S22.

In the next step S22, the angle deviation detector 30 calculates the difference (i.e., the second difference) in rotation angle between the first rotating frame 15A and the second rotating frame 15B on the basis of the respective rotation angles (i.e., absolute positions) detected by the first angle sensor 18A and the second angle sensor 18B.

In the next step S23, the angle deviation detector 30 determines whether the calculated difference exceeds the predetermined threshold (i.e., the second threshold) or not. If the difference does not exceed the predetermined threshold, i.e., the second threshold (NO in the step S23), the angle-deviation detection processing is completed. If the difference exceeds the predetermined threshold, i.e., the second threshold (YES in the step S23), the processing proceeds to the step S24.

In the next step S24, the angle deviation detector 30 executes emergency stop processing by which rotation of the rotating frames 15 in rotating motion is stopped.

Since the angle deviation detector 30 is provided in the second embodiment, damage to the equipment caused by the left and right angle deviation of the rotating frames 15 during synchronization control can be avoided.

Since the correction-value table updaters 27 are provided, the correction-value table is updated every time the system is driven and can be kept up to date, which eliminates the need for users to separately perform a test for updating the correction-value table.

### Third Embodiment

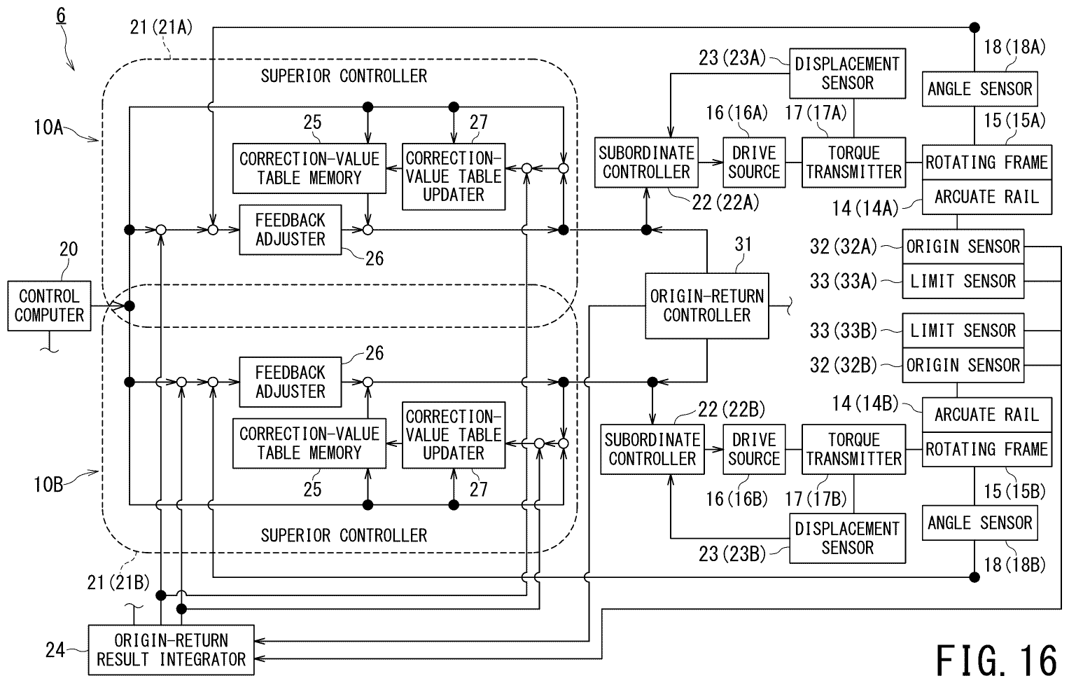

Next, the third embodiment will be described by referring to FIG. 16 to FIG. 21. The same components as those shown in the above-described embodiments are denoted by the same reference signs, and duplicate descriptions are omitted.

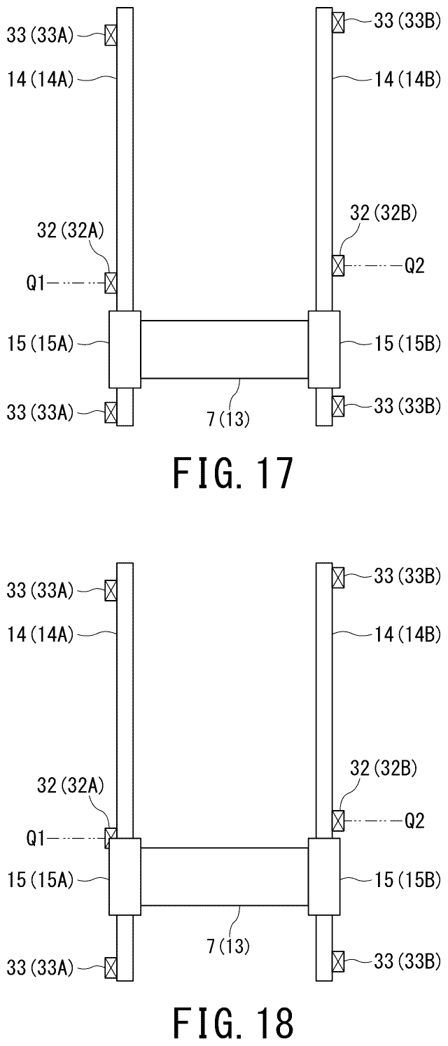

As shown in FIG. 16, each superior controller 21 according to the third embodiment is provided with the feedback adjuster 26 and the correction-value table updater 27, similarly to the configuration of the above-described second embodiment. The synchronization control apparatus 6 according to the third embodiment further includes an origin-return controller 31, two origin sensors 32 (FIG. 17), and two pairs of limit sensors 33 (FIG. 17) in addition to the components of the above-described first embodiment.

In the following description, the members and components corresponding to the first unit 10A are sometimes referred to as the first origin sensor 32A and the first limit sensors 33A, and the members and components corresponding to the second unit 10B are sometimes referred to as the second origin sensor 32B and the second limit sensors 33B.

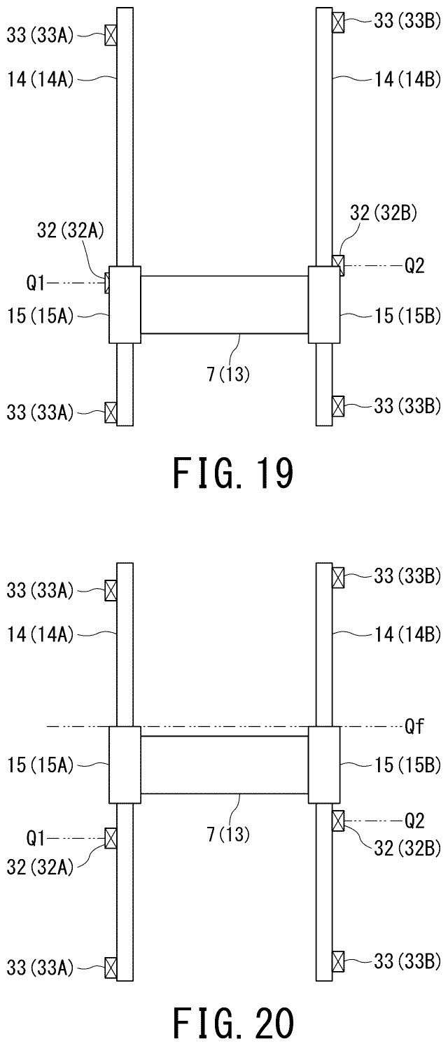

As shown in FIG. 17, the origin sensors 32 are provided on the respective arcuate rails 14. Each origin sensor 32 detects the upper end of the corresponding rotating frame 15 when this rotating frame 15 reaches the mechanical origin specific to the apparatus. For example, as shown in FIG. 18, when the upper end of the first rotating frame 15A reaches the first origin Q1 (i.e., mechanical origin) in the first arcuate rail 14A, the first origin sensor 32A detects this positional attainment. As shown in FIG. 19, when the upper end of the second rotating frame 15B reaches the second origin Q2 (i.e., mechanical origin) in the second arcuate rail 14B, the second origin sensor 32B detects this positional attainment.

As shown in FIG. 16, the origin-return controller 31 performs origin-return control by which each rotating frame 15 is caused to reach the mechanical origin. The origin-return controller 31 is connected to the control computer 20 and is controlled by the control computer 20.

The origin-return result integrator 24 according to the third embodiment detects the positional deviation between the mechanical origin and the position of the actual rotation angle (i.e., absolute position) detected by the angle sensors 18 when the rotating frames 15 are returned to the origin. For example, the origin offset is sometimes performed in such a manner that an arbitrarily settable software origin Qf (i.e., control origin) is set by the user at a position different from the first origin Q1 (i.e., mechanical origin) and the second origin Q2 (i.e., mechanical origin). Here, there may be a case where the first origin Q1 and the second origin Q2 are misaligned. The origin-return result integrator 24 performs processing of adding the origin-offset value and the positional deviation value to the target-position command value when this origin offset is performed.

As shown in FIG. 17, the limit sensors 33 are provided on the side surface of each of the arcuate rails 14. Each limit sensor 33 detects the upper or lower end of the corresponding rotating frame 15 when this rotating frame reaches the end of its rotatable range. One pair of limit sensors 33 indicate the range of motion boundaries of the corresponding rotating frame 15. For example, the first arcuate rail 14A is provided with a pair of upper and lower first limit sensors 33A near one end and the other end. In addition, the second arcuate rail 14B is provided with a pair of upper and lower second limit sensors 33B near one end and the other end.

The installation work of the origin sensors 32 and the limit sensors 33 with respect to the arcuate rails 14 is manually performed by a worker, so the installation positions of these sensors on the left and right may be slightly misaligned. For example, the first origin Q1 to be defined by the first origin sensor 32A on the first arcuate rail 14A and the second origin Q2 to be defined by the second origin sensor 32B on the second arcuate rail 14B may not necessarily be same angle position and may be misaligned. Similarly, the installation positions of the pair of first limit sensors 33A are not necessarily same angle positions to the installation positions of the pair of second limit sensors 33B and may be misaligned. For this reason, origin return processing is performed in the third embodiment.

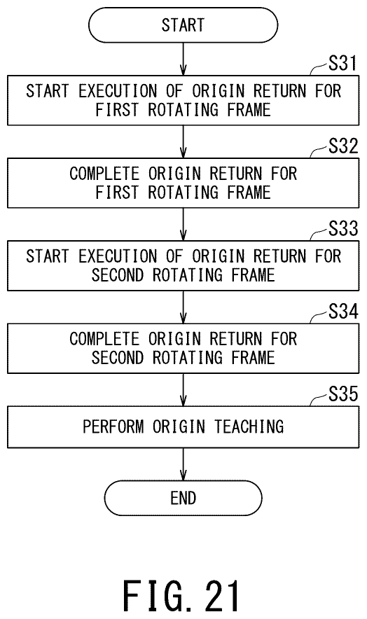

Next, the origin return processing will be described on the basis of the flowchart of FIG. 21 by referring to the block diagram of FIG. 16. The following steps are at least part of the origin return processing, and other steps may be included in the origin return processing.

Although the first rotating frame 15A and the second rotating frame 15B always operate synchronously during the origin return processing, these rotating frames 15 return to their origin individually or in a time-division manner during the origin return processing. For example, the first rotating frame 15A is returned to its origin first, and then the second rotating frame 15B is returned to its origin.

In the first step S31, the origin-return controller 31 inputs the target-position command value, which sets the first origin Q1 as the target position, into the first subordinate controller 22A and the second subordinate controller 22B. Subsequently, processing of returning the first rotating frame 15A to the origin begins. The first rotating frame 15A and the second rotating frame 15B move to the position of the first origin Q1 (FIG. 18).

In the next step S32, at the time at which the first rotating frame 15A and the second rotating frame 15B reach the first origin Q1 (FIG. 18), the origin-return controller 31 completes the origin return of the first rotating frame 15A.

In the next step S33, the origin-return controller 31 inputs the target-position command value, which sets the second origin Q2 as the target position, into the first subordinate controller 22A and the second subordinate controller 22B. Subsequently, processing of returning the second rotating frame 15B to the origin begins. The first rotating frame 15A and the second rotating frame 15B move toward the position of the second origin Q2 (FIG. 19).

In the next step S34, at the time at which the first rotating frame 15A and the second rotating frame 15B reach the second origin Q2 (FIG. 19), the origin-return controller 31 completes the origin return of the second rotating frame 15B.

In the next step S35, the origin-return controller 31 performs origin teaching. Here, the software origin Qf for the target-position command value of the rotating frames 15 is set (FIG. 20). For example, the origin-return result integrator 24 sets the value obtained by adding the origin-offset value to the first origin Q1 as the software origin Qf, and sets the value obtained by adding the origin-offset value to the second origin Q2 as the software origin Qf.

Each correction-value table updater 27 updates the correction-value table on the basis of the origin-offset value obtained by the origin-return result integrator 24.

In the third embodiment, the origin-offset value and the positional deviation value can be reflected in the target-position command value, which enables accurate positioning of the irradiation port 7.

As above, although the present invention has been described on the basis of the first to third embodiments, the configuration applied in any one of the embodiments may be applied to other embodiments or the configurations in the respective embodiments may be applied in combination.

In the above-described embodiments, the determination of one target value (i.e., the first difference and the second difference) using a reference value (i.e., the range, the threshold) may be determination of whether the target value is equal to or larger than the reference value or not. Additionally, or alternatively, the determination of the target value using the reference value may be determination of whether the target value exceeds the reference value or not. Additionally, or alternatively, the determination of the target value using the reference value may be determination of whether the target value is equal to or smaller than the reference value or not. Additionally, or alternatively, the determination of the one value using the reference value may be determination of whether the target value is smaller than the reference value or not. Additionally, or alternatively, the reference value is not necessarily fixed, and the reference value may be changed. Thus, a predetermined range of values may be used instead of the reference value, and the determination of the target value may be determination of whether the target value is within the predetermined range or not. In addition, an error occurring in the apparatus may be analyzed in advance, and the predetermined range including the error range centered on the reference value may be used for determination.

Although a mode in which each step is executed in series is illustrated in the flowcharts of the above-described embodiments, the execution order of the respective steps is not necessarily fixed and the execution order of part of the steps may be changed. Additionally, some steps may be executed in parallel with another step.

Note that the program executed in the synchronization control apparatus 6 is provided by being incorporated in a memory such as the ROM in advance. Additionally, or alternatively, the program may be provided by being stored as a file of installable or executable format in a non-transitory computer-readable storage medium such as a CD-ROM, a CD-R, a memory card, a DVD, and a flexible disk (FD).

In addition, the program executed in the synchronization control apparatus 6 may be stored on a computer connected to a network such as the Internet and be provided by being downloaded via a network. That is, the program may be provided from resources of cloud computing. Alternatively, a server on the cloud computing may execute the program and only its processing result may be provided via the cloud computing.

The number of members and devices provided in the synchronization control apparatus 6 is not limited to the aspect of the above-describe embodiments and may be changed as appropriate. For example, the number of the arcuate rails 14 is not limited to two but may be three or more. In addition, the number of the superior controllers 21 is not limited to two but may be three or more.

According to at least one embodiment described above, each superior controller 21 corrects the target-position command value on the basis of the correction-value table and outputs the corrected target-position command value to the corresponding subordinate controller 22. This control allows both rotating frames 15 for supporting a heavy load to be synchronously driven and be positioned with high precision when driving both rotating frames.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. The articles “the”, “a” and “an” are not necessarily limited to mean only one, but rather are inclusive and open ended so as to include, optionally, multiple such elements.

What is claimed is:

1. A synchronization control apparatus comprising:
at least two arcuate rails fixedly provided in an arc-shape and arranged in such a manner that arcs of respective arcuate rails are parallel to each other;
at least two rotating frames corresponding to the respective arcuate rails, each of the rotating frames being configured to rotationally move along a corresponding arcuate rail and around a center axis of the corresponding arcuate rail and support a support subject;
at least two drive sources configured to provide torque for rotationally moving respective rotating frames;
at least two torque transmitters provided in respective drive sources and configured to transmit the torque to the respective rotating frames;
at least two displacement sensors provided in respective torque transmitters and configured to measure displacement amounts of respective components to be displaced by transmission of the torque, the respective components constituting the torque transmitters;
at least two subordinate controllers configured to control the respective drive sources while feeding back respective displacement amounts measured by the displacement sensors in such a manner that each of the rotating frames reaches a position of a target rotation angle indicating an inputted target-position command value; and
at least two superior controllers corresponding to respective subordinate controllers, each of the superior controllers being configured to correct the target-position command value by using a correction-value table and output a corrected target-position command value to a corresponding subordinate controller, the correction-value table being a table in which correction values for correcting the target-position command value are registered in advance.

2. The synchronization control apparatus according to claim 1, wherein:
each of the arcuate rails and the rotating frames forms a C-shape; and
opening dimensions of a cut-out portion of each of the arcuate rails and the rotating frames satisfy a condition that an object can enter from a direction intersecting a center axis of each of the arcuate rails in a case of disposing the object near the support subject.

3. The synchronization control apparatus according to claim 1, further comprising:
at least two angle sensors provided in the respective rotating frames, each of the angle sensors being configured to detect a tilt of a corresponding rotating frame, the tilt being a rotation angle when the corresponding rotating frame rotates;
at least two feedback adjusters corresponding to the respective rotating frames, each of the feedback adjuster being configured to perform adjustment of the target-position command value of a corresponding rotating frame on a premise that there is a difference between the rotation angle targeted by the target-position command value and an actual rotation angle detected by a corresponding angle sensor, the adjustment being performed in such a manner that the difference falls within a predetermined range; and
at least two correction-value table updaters corresponding to the respective rotating frames, each of the correction-value table updaters being configured to update the correction value registered in the correction-value table of a corresponding rotating frame by using the target-position command value adjusted when the difference falls within the predetermined range.

4. The synchronization control apparatus according to claim 1, further comprising:
at least two angle sensors provided in the respective rotating frames, each of the angle sensors being configured to detect a tilt of a corresponding rotating frame, the tilt being a rotation angle when the corresponding rotating frame rotates; and
an angle deviation detector configured to:
calculate a difference in rotation angle between one of the rotating frames and another of the rotating frames depending on respective rotation angles detected by the angle sensors during rotation of the rotating frames, and
stop the rotation of the rotating frames when the difference exceeds a predetermined threshold.

5. The synchronization control apparatus according to claim 1, further comprising an origin-return result integrator configured to add an origin-offset value to the target-position command value when origin offset of setting an arbitrarily settable control origin at a position different from a mechanical origin specific to an apparatus is performed by a user.

6. The synchronization control apparatus according to claim 1, further comprising:
at least two angle sensors provided in the respective rotating frames, each of the angle sensors being configured to detect a tilt of a corresponding rotating frame, the tilt being a rotation angle when the corresponding rotating frame rotates;
at least two origin sensors provided in the respective arcuate rails, each of the origin sensors being configured to detect positional attainment when a corresponding rotating frame reaches a mechanical origin specific to an apparatus;
an origin-return controller configured to perform origin-return control by which each of the rotating frames is returned to the mechanical origin; and
an origin-return result integrator configured to:
detect positional deviation between the mechanical origin and a position of an actual rotation angle detected by each of the angle sensors on a fact that each of the rotating frames is returned to an origin, and
add an origin-offset value and a value of the positional deviation to the target-position command value on a fact that origin offset of setting an arbitrarily settable control origin at a position different from the mechanical origin is performed by a user.

7. A synchronization control method of using:
at least two arcuate rails fixedly provided in an arc-shape and arranged in such a manner that arcs of respective arcuate rails are parallel to each other;
at least two rotating frames corresponding to the respective arcuate rails, each of the rotating frames being configured to rotationally move along a corresponding arcuate rail and around a center axis of the corresponding arcuate rail and support a support subject;
at least two drive sources configured to provide torque for rotationally moving respective rotating frames;
at least two torque transmitters provided in respective drive sources and configured to transmit the torque to the respective rotating frames;
at least two displacement sensors provided in respective torque transmitters and configured to measure displacement amounts of respective components to be displaced by transmission of the torque, the respective components constituting the torque transmitters;
at least two subordinate controllers configured to control the respective drive sources while feeding back respective displacement amounts measured by the displacement sensors in such a manner that each of the rotating frames reaches a position of a target rotation angle indicating an inputted target-position command value; and
at least two superior controllers corresponding to respective subordinate controllers, each of the superior controllers being configured to output the target-position command value to a corresponding subordinate controller,
the synchronization control method comprising steps of:
causing each of the superior controllers to correct the target-position command value by using a correction-value table in which correction values for correcting the target-position command value are registered in advance; and
causing each of the superior controllers to output a corrected target-position command value to the corresponding subordinate controller.

8. A heavy particle beam irradiation system comprising:
the synchronization control apparatus according to claim 1;
an ion generator configured to generate a heavy particle beam;
an accelerator configured to accelerate the heavy particle beam generated by the ion generator; and
an irradiation port configured to irradiate an irradiation target with the heavy particle beam accelerated by the accelerator and be supported as the support subject by the rotating frames.
